(19) United States
(12) Patent Application Publication
(10) Pub. No.: US 2021/0363593 A1
(43) Pub. Date: Nov. 25, 2021

(54) CXCL13 Marker For Predicting Immunotherapeutic Responsiveness In Patient With Lung Cancer And Use Thereof

(71) Applicant: SAMSUNG LIFE PUBLIC WELFARE FOUNDATION, Seoul (KR)

(72) Inventors: Se Hoon LEE, Seoul (KR); Sehhoon PARK, Seoul (KR); Hong Sook KIM, Gyeonggi-do (KR); Hongui CHA, Seoul (KR)

(21) Appl. No.: 17/254,977

(22) PCT Filed: Oct. 28, 2020
(86) PCT No.: PCT/KR2020/014823

§ 371 Date: Dec. 22, 2020

(30) Foreign Application Priority Data
Oct. 30, 2019 (KR) 10-2019-0136695

(51) Int. Cl. C12Q 1/6886; C12Q 1/686

(52) U.S. Cl. CPC C12Q 1/6886; C12Q 1/686; C12Q 2600/158; C12Q 2600/136; C12Q 2600/106

(57) ABSTRACT

The present invention relates to a biomarker for predicting immunotherapy responsiveness in a patient with lung cancer and a use thereof, and more specifically, to a marker composition for predicting immunotherapy responsiveness in a patient with lung adenocarcinoma, a composition for predicting immunotherapy responsiveness, and a method for providing information for predicting immunotherapy responsiveness, comprising a CXCL13 gene or a protein encoded by the gene, and it was confirmed that CXCL3 was significantly upregulated in a patient administered an immune checkpoint inhibitor using the composition of the present invention, and through this, the CXCL13 gene or the protein encoded by the gene may be usefully used as a marker for predicting anticancer drug therapy responsiveness against lung cancer in related research fields, and is expected to be utilized as an immunotherapeutic adjuvant which enhances the anticancer treatment effect by developing an enhancer.

### FIELD

The present invention relates to a biomarker for predicting immunotherapeutic responsiveness in a patient with lung cancer and a use thereof, and more specifically, to a marker composition for predicting immunotherapeutic responsiveness in a patient with lung adenocarcinoma, a composition for predicting immunotherapeutic responsiveness, and a method for providing information for predicting immunotherapeutic responsiveness, comprising a CXCL13 gene or a protein encoded by the gene.

### BACKGROUND

Lung cancer, which is a malignant tumor originating in the lungs, may be roughly divided into small cell lung cancer and non-small cell lung cancer according to the tissue morphology thereof. Non-small cell lung cancer is classified into adenocarcinoma, squamous cell carcinoma, and large-cell carcinoma according to tissue type.

Non-small cell lung cancer is classified into adenocarcinoma, squamous cell carcinoma, and large-cell carcinoma, as described above. First, adenocarcinoma occurs predominantly in the peripheral lung sites, occurs frequently in women or non-smokers, metastasizes in many cases despite the small sizes thereof, and the occurrence frequency thereof has tended to increase recently. Next, squamous cell carcinoma is found predominantly in the central part of the lungs, and shows symptoms that usually extend to the bronchial lumen and block the bronchi, and it is known that squamous cell carcinoma is common in men and is closely related to smoking. Finally, large-cell carcinoma occurs predominantly near the surface of the lungs, about half of which occurs in the large bronchi, accounts for about 4 to 10% of all lung cancers, and is predominantly large in size, some of large-cell carcinoma tends to grow and metastasize rapidly, so that the prognosis is known to be poorer than other non-small cell lung cancers.

In particular, in the case of non-small cell lung cancer, the survival rate for the last 10 years is only about 10%, and one of the main reasons for such a low survival rate is that the success rate of lung cancer diagnosis is very low. As a method capable of improving the success rate of the lung cancer diagnosis, a method for detecting a gene abnormality specific to lung cancer has been proposed, and for this purpose, various research results on lung cancer-specific gene mutations have been reported, but there is a disadvantage in that the criteria for determining whether or not such a gene is abnormal are ambiguous in the early stage of lung cancer progression. That is, the correlation between the mutation level of a specific gene and the likelihood of getting lung cancer is not clear, so that the clear criteria for suspected lung cancer onset when various lung cancer-related genes are mutated are ambiguous, and these problems occur in the diagnosis of not only in lung cancer but also all other cancers.

Although studies have been conducted on markers used for the detection or diagnosis of human lung adenocarcinoma (Korean Patent No. 1064561, and the like), there is a still insufficient research conducted on the prediction of immunotherapeutic responsiveness of a lung cancer patient including a CXCL13 gene or a protein encoded by the gene.

### SUMMARY

### Technical Problem

As a result of studies conducted to discover a biomarker for predicting immunotherapeutic responsiveness in a patient with lung cancer, the present inventors derived an immunotherapy response biomarker according to the present invention by confirming that high expression of CXCL affected the local regional immunity of tumors based on the correlation between the expression of CXCL13 and the formation of tertiary lymphoid structures (TLS), and analyzing expression profiles of CXCL13 and an immune-related gene to confirm a gene differentially expressed in lung cancer, thereby completing the present invention based on this.

Thus, an object of the present invention is to provide a marker composition for predicting immunotherapeutic responsiveness in a patient with lung cancer, including a CXCL13 gene or a protein encoded by the gene.

Further, another object of the present invention is to provide a composition for predicting immunotherapeutic responsiveness in a patient with lung cancer, including a preparation which measures the level of mRNA of a CXCL13 gene or a protein encoded by the gene.

In addition, still another object of the present invention is to provide a kit for predicting immunotherapeutic responsiveness, including the composition.

Furthermore, yet another object of the present invention is to provide a method for providing information for predicting immunotherapeutic responsiveness in a patient with lung cancer, the method including measuring the expression level of mRNA of a CXCL13 gene or a protein encoded by the gene for a subject-derived biological sample.

Further, yet another object of the present invention is to provide an immunotherapeutic adjuvant including an agent for enhancing the expression of a CXCL13 gene or the activity of a protein thereof.

In addition, yet another object of the present invention is to provide a method for screening an immunotherapeutic adjuvant, the method including: (a) treating cells in vivo with a candidate material; (b) selecting a candidate material in which the expression of mRNA of a CXCL13 gene or a protein thereof is increased compared to a candidate material non-treatment group by measuring the expression level of mRNA of the CXCL13 gene or the protein thereof; and (c) selecting the selected candidate material as an immunotherapeutic adjuvant.

However, technical problems to be achieved by the present invention are not limited to the aforementioned problems, and other problems that are not mentioned may be clearly understood by those skilled in the art from the following description.

### Technical Solution

In order to achieve the aforementioned objects of the present invention, the present invention provides a marker composition for predicting immunotherapeutic responsiveness in a patient with lung cancer, including a CXCL13 gene or a protein encoded by the gene.

As an exemplary embodiment of the present invention, the immunotherapy may be treatment with a PD-1 inhibitor or a PDL-1 inhibitor.

As another exemplary embodiment of the present invention, the lung cancer may be lung adenocarcinoma.

Further, the present invention provides a composition for predicting immunotherapeutic responsiveness in a patient with lung cancer, including a preparation which measures the level of mRNA of a CXCL13 gene or a protein encoded by the gene.

As an exemplary embodiment of the present invention, the preparation which measures the level of mRNA of the gene may be a set of sense and antisense primers or a probe which binds complementarily to the mRNA of the gene.

As another exemplary embodiment of the present invention, the preparation which measures the level of the protein may be an antibody which binds specifically to the protein encoded by the gene.

In addition, the present invention provides a kit for predicting immunotherapeutic responsiveness, including the composition.

Furthermore, the present invention provides a method for providing information for predicting immunotherapeutic responsiveness in a patient with lung cancer, the method including measuring the expression level of mRNA of a CXCL13 gene ora protein encoded by the gene for a subject-derived biological sample.

As an exemplary embodiment of the present invention, the expression level of mRNA may be measured by one or more methods selected from the group consisting of NanoString nCounter analysis, a polymerase chain reaction (PCR), a reverse transcription-polymerase chain reaction (RT-PCR), a real-time polymerase chain reaction (Real-time PCR), RNase protection assay (RPA), a microarray, and northern blotting.

As another exemplary embodiment of the present invention, the expression level of the protein may be measured by one or more methods selected from the group consisting of western blotting, radioimmunoassay (RIA), radioimmunodiffusion, enzyme-linked immunosorbent assay (ELISA), immunoprecipitation, flow cytometry, immunofluorescence, Ouchterlony double immunodiffusion, complement fixation assay, and a protein chip.

As still another exemplary embodiment of the present invention, the biological sample may be a tissue derived from a patient with lung cancer.

Further, the present invention provides an immunotherapeutic adjuvant including an agent for enhancing the expression of a CXCL13 gene or the activity of a protein thereof,

In addition, the present invention provides a method for screening an immunotherapeutic adjuvant, the method including: (a) treating cells in vivo with a candidate material; (b) selecting a candidate material in which the expression of mRNA of a CXCL13 gene or a protein thereof is increased compared to a candidate material non-treatment group by measuring the expression level of mRNA of the CXCL13 gene or the protein thereof; and (c) selecting the selected candidate material as an immunotherapeutic adjuvant.

As an exemplary embodiment of the present invention, the candidate material may be selected from the group consisting of nucleic acids, compounds, microbial culture solutions or extracts, natural product extracts, peptides, substrate analogues, aptamers, and antibodies.

Furthermore, the present invention provides a composition for predicting the survival rate prognosis of a patient with lung cancer, including a CXCL13 gene or a protein encoded by the gene.

Further, the present invention provides a use of the composition for predicting an immunotherapy response in a patient with lung cancer.

In addition, the present invention provides a use of the composition for predicting a survival rate prognosis in a patient with lung cancer.

### Advantageous Effects

By confirming that CXCL13 was significantly upregulated in a patient administered an immune checkpoint inhibitor using the composition according to the present invention, a CXCL13 gene or a protein encoded by the gene can be usefully used as marker for predicting anticancer drug therapeutic responsiveness against lung cancer in related research fields. Furthermore, it is expected that it is possible to develop a new immunotherapeutic adjuvant, which can enhance an anticancer immunotherapeutic effect in a patient with lung cancer by regulating the level of mRNA of a CXCL13 gene or a protein encoded by the gene.

### BRIEF DESCRIPTION OF DRAWINGS

FIG. 1A illustrates a Volcano plot of a differentially expressed gene showing a significant change between response groups and non-response groups to an immune checkpoint inhibitor in a search data set (n=63).

FIG. 1B illustrates a heatmap of representative genes exhibiting a fold change value of more than 1.5 and a P-value of more than 0.05.

FIG. 1C illustrates the TPM and tumor purity of CXCL13 calculated by ESTIMATE.

FIG. 1D illustrates the TPM of CXCL13 based on the exhibition of the highest response to an immune checkpoint inhibitor (ICI).

FIG. 1E illustrates the predicted value of CXCL13 for an ICI, and FIG. 1F illustrates the results of confirming disease-free survival and overall survival using the median cut-off value of the expression profile of CXCL13.

FIG. 2A illustrates the correlation between the expression profile of CXCL13 and representative genes related to cytotoxic activity.

FIG. 2B illustrates a heatmap of gene sets related to a tertiary lymphoid structure (TLS).

FIG. 2C illustrates the correlation between 22C3PharDx PD-L1 immunohistochemical expression and CXCL13 expression (n=51) whose P-value was measured by the Kruskal-Wallis test.

FIG. 2D illustrates the correlation between PDCD1 and CD274, which exhibit the expression of CXCL13.

FIG. 2E illustrates the correlation between tumor mutations and CXCL13 in samples available for somatic mutation profiles (n=40) except for one or more values.

FIG. 2F illustrates a survival analysis based on the expression of CXCL13 and CD103.

FIG. 2G illustrates the expression of CXCL13 and CD8A using the median TPM as a cut-off value.

FIG. 3A illustrates a Volcano plot of a differentially expressed gene showing a significant change between response groups and non-response groups to an immune checkpoint inhibitor in a verification data set (n=57).

FIG. 3B illustrates the difference in expression of CXCL13 whose P-value was measured by the Mann-Whitney test.

FIG. 3C illustrates the results of confirming disease-free survival and overall survival using the median cut-off value of the expression profile of the CXCL13.

FIG. 3D illustrates the difference in CXCL13 profile expression between normal tissues and lung adenocarcinoma tissues through TCGA data.

FIG. 3E illustrates the results of confirming that there is no difference in overall survival based on the CXCL13 expression profile.

FIG. 3F illustrates the results of confirming drug reactivity by expressing CXCL13 using a data set independent of renal cell carcinoma and melanoma.

FIG. 3G illustrates the results of confirming survival prediction by expressing CXCL13 using a data set independent of renal cell carcinoma and melanoma.

FIG. 4A illustrates the predicted values of multiple gene sets in the search data set.

FIG. 4B illustrates the predicted values of the CTL gene set and GEP gene set combined with CXCL13.

FIG. 5 illustrates the expression profiles of cytotoxic activity genes CCL19, CCL21, ENTPD1, CXCL12, ITGAE, GZMA, GZMB, CD8A, CXCL9, CD8B, IFNG, CD79B, and PRF1 known to be associated with immune checkpoint inhibitor reactivity based on the immune checkpoint inhibitor response of the search data set.

FIG. 6A illustrates a heatmap in which genes having a fold change value in expression profile exceeding 1.5 and a P-value exceeding 0.05 are confirmed in the response groups compared to the non-response groups.

FIG. 6B illustrates the distribution of CXCL13 expression and tumor purity based on a biopsy site.

FIG. 6C illustrates predicted values for the immune inhibitor response of CXCL3 TPM values.

FIG. 6D illustrates the correlation of CXCL13 with a tumor mutation burden.

FIG. 6E illustrates the correlation of CXCL13 with PD-L1 immunohistochemistry.

FIG. 6F illustrates the interaction of CXCL13 with the TPM values of PDCD1 and CD274.

FIG. 7A illustrates a heatmap of CXCL13 and gene sets related to TLS.

FIG. 7B illustrates the correlation between CXCL13 and a gene of interest.

FIG. 8 illustrates the expression profiles of cytotoxic activity genes CCL19, CCL21, ENTPD1, CXCL12, ITGAE, GZMA, GZMB, CD8A, CXCL9, CD8B, IFNG, CD79B, and PRF1 known to be associated with immune checkpoint inhibitor reactivity based on the immune checkpoint inhibitor response of the verification data set.

FIG. 9 illustrates the correlation between the CXCL13 expression profile and representative genes associated with cytotoxic activity in TCGA lung adenocarcinoma samples not treated with immuno-oncology agents.

FIG. 10A illustrates the correlation between CXCL13 expression and representative genes associated with independent cytotoxic activity in the renal cell carcinoma population.

FIG. 10B illustrates the correlation between CXCL13 expression and representative genes associated with independent cytotoxic activity in the melanoma cell population.

### DETAILED DESCRIPTION

As a result of studies conducted to discover a biomarker for predicting immunotherapeutic responsiveness in a patient with lung cancer, the present inventors derived an immunotherapy response biomarker according to the present invention by confirming that high expression of CXCL affected a local regional immunity of tumors based on the correlation between the expression of CXCL13 and the formation of tertiary lymphoid structures (TLS), and analyzing expression profiles of CXCL13 and an immune-related gene using a T-test method to confirm a gene differentially expressed in lung cancer and the effectiveness of the gene, thereby completing the present invention based on this.

Thus, the present invention provides a marker composition for predicting immunotherapeutic responsiveness in a patient with lung cancer, including a CXCL13 gene or a protein encoded by the gene, a composition for predicting immunotherapeutic responsiveness in a patient with lung cancer, including a preparation which measures the level of m RNA of a CXCL13 gene or a protein encoded by the gene, and a kit for predicting anticancer drug therapeutic responsiveness, including the composition.

A C-X-C motif chemokine ligand 13 (CXCL13) according to the present invention may include an amino acid sequence of SEQ ID NO: 1, but is not limited thereto, and it is possible to use an amino acid sequence obtained by deleting, adding, or substituting some amino acids in the aforementioned amino acid sequence, and a sequence having specifically 80% or more, more specifically 90% or more, and most preferably 95%, 96%, 97%, 98%, and 99% homology with the aforementioned amino acid sequence.

Further, a CXCL13 gene (NM_06419.2, NM_001371558.1) according to the present invention may include a base sequence of SEQ ID NO: 2 or SEQ ID NO: 3.

As an exemplary embodiment of the present invention, the immunotherapy may be treatment with a PD-1 inhibitor or a PDL-1 inhibitor, and may be selected from sipuleucel-T, ipilimumab, pembrolizumab, nivolumab, atezolizumab, durvalumab, talimogene laherparepvec, and tisagenlecleucel, and is preferably treatment with pembrolizumab, nivolumab, or atezolizumab, but is not limited thereto.

As another exemplary embodiment of the present invention, the lung cancer may be selected from the group consisting of small cell lung cancer and non-small cell lung cancer, specifically, adenocarcinoma, squamous cell carcinoma, and large-cell carcinoma, which are non-small cell lung cancers, and more specifically, lung adenocarcinoma is more preferred.

As used herein, the term “immunotherapy” refers to a method of stimulating the immune system to treat a disease, and refers to the treatment of lung cancer in the present invention. Passive immunotherapy is a treatment method in which a large amount of immune response components produced in vitro, such as immune cells, antibodies, and cytokines, are injected into a patient with cancer to attack the cancer cells, and active immunotherapy is a treatment method which actively activates or produces an individual's antibodies and immune cells to attack cancer cells. The present invention relates to a biomarker for predicting the therapeutic responsiveness of a patient with lung cancer to such immunotherapy and a use thereof.

As used herein, “prediction of therapeutic responsiveness” refers to predicting whether or not a patient responds favorably or unfavorably to an immune anticancer drug, or predicting the risk of resistance to an anticancer drug, and predicting the prognosis of the patient after immunotherapy, that is, recurrence, metastasis, survival, or disease-free survival. The biomarker for predicting therapeutic responsiveness according to the present invention may provide information for selecting the most appropriate immunotherapy method for a patient with lung cancer.

As an exemplary embodiment of the present invention, the preparation which measures the level of mRNA of the CXCL13 gene may be a set of sense and antisense primers or a probe which binds complementarily to the mRNA of the gene, but is not limited thereto.

As used herein, the term “primer” refers to an oligonucleotide synthesized for the purpose of being used for diagnosis, DNA sequencing, and the like as a short gene sequence which becomes an origin of the DNA synthesis. The primers may be typically synthesized to a length of 15 to 30 base pairs and used, but may vary depending on the purpose of use, and may be modified by methylation and capping by a known method.

As used herein, the term “probe” refers to a nucleic acid capable of binding specifically to an mRNA having several to several hundred bases constructed via enzyme chemical separation or purification or a synthesis process. The probe can identify the presence or absence of mRNA by labeling a radioactive isotope, an enzyme, a fluorescent material, or the like, and may be designed, modified by a known method, and used.

As another exemplary embodiment of the present invention, the preparation which measures the level of the protein may be an antibody which binds specifically to the protein encoded by the gene, but is not limited thereto.

As used herein, the term “antibody” includes an immunoglobulin molecule having immunological reactivity with a certain antigen, and includes both a monoclonal antibody and a polyclonal antibody. Furthermore, the antibody includes a form produced by genetic engineering, such as a chimeric antibody (for example, a humanized murine antibody) and an antibody binding to two different types of antigen (for example, a bispecific antibody).

As still another exemplary embodiment of the present invention, the kit for predicting immunotherapeutic responsiveness may include one or more other constituent component compositions, solutions or devices suitable for an analysis method.

As another aspect of the present invention provides a method for providing information for predicting immunotherapeutic responsiveness in a patient with lung cancer, the method including measuring the level of mRNA of a CXCL13 gene or a protein encoded by the gene in a subject-derived biological sample.

As used herein, the term “method for providing information for predicting immunotherapeutic responsiveness in a patient with lung cancer” means to provide objective basic information necessary for diagnosing a lung cancer disease as a preliminary step for diagnosis or prognosis prediction, and excludes the physician's clinical judgment or findings. The subject-derived biological sample is not limited to, but may be, for example, a tissue, a cell, and the like, and is preferably a tissue or a cell derived from a patient with lung cancer.

In the present invention, the expression level of mRNA may be measured by one or more methods selected from the group consisting of NanoString nCounter analysis, a polymerase chain reaction (PCR), a reverse transcription-polymerase chain reaction (RT-PCR), a real-time polymerase chain reaction (Real-time PCR), RNase protection assay (RPA), a microarray, and northern blotting according to a typical method known in the art, but is not limited thereto.

In the present invention, the expression level of the protein may be measured by one or more methods selected from the group consisting of western blotting, radioimmunoassay (RIA), radioimmunodiffusion, enzyme-linked immunosorbent assay (ELISA), immunoprecipitation, flow cytometry, immunofluorescence, Ouchterlony double immmunodiffusion, complement fixation assay, and a protein chip according to a typical method known in the art, but is not limited thereto.

The present inventors have confirmed the effectiveness for predicting immunotherapeutic responsiveness including the CXCL13 gene or the protein encoded by the gene.

In an exemplary embodiment of the present invention, it was confirmed that 21 genes were upregulated in responsive patients by comparing the differentially expressed gene (DEG) profile with non-responsive patients to confirm the upregulation of CXCL13 of the search data set in ICI patients.(see Example 3).

Through the results, the composition including the CXCL13 gene or the protein encoded by the gene of the present invention may be usefully used for predicting immunotherapeutic responsiveness in a patient with lung cancer.

As still another aspect of the present invention, the present invention provides an immunotherapeutic adjuvant including an agent for enhancing the expression of a CXCL13 gene or the activity of a protein thereof.

As yet another aspect of the present invention, the present invention provides a method for screening an immunotherapeutic adjuvant, the method including: (a) treating cells in vivo with a candidate material; (b) selecting a candidate material in which the expression of mRNA of a CXCL13 gene or a protein thereof is increased compared to a candidate material non-treatment group by measuring the expression level of mRNA of the CXCL13 gene or the protein thereof; and (c) selecting the selected candidate material as an immunotherapeutic adjuvant.

In the present invention, the candidate material may be selected from the group consisting of nucleic acids, compounds, microbial culture solutions or extracts, natural product extracts, peptides, substrate analogues, aptamers, and antibodies, and the nucleic acid may be preferably selected from the group consisting of siRNA, shRNA, microRNA, antisense RNA, a locked nucleic acid (LNA), a peptide nucleic acid (PNA), and a morpholino, but is not limited thereto.

As yet another aspect of the present invention, the present invention provides a composition for predicting the survival rate prognosis of a patent with lung cancer, including a CXCL13 gene or a protein encoded by the gene.

As used herein, the term “survival rate prognosis” is associated with whether or not a patient survives or the possibility that the patient survives, after treatment of the patient by responding favorably or unfavorably to a treatment method such as chemotherapy, for example, treatment by chemotherapy for a specific period of time.

The predictive marker compositions of the present invention may be used clinically to make treatment decisions by selecting the most appropriate treatment method for a patient with lung cancer. In addition, the predictive composition of the present invention may confirm whether the patient responds favorably to a treatment regimen such as a predetermined treatment regimen, for example, administration of a predetermined therapeutic agent or combination, surgical intervention, chemotherapy, and the like, or predict whether a patient can survive for a long period of time after the treatment regimen.

Through the examples, the present inventors confirmed that the survival prognosis of a patient with lung cancer can be predicted by the expression level of the chemokine CXCL13 according to the present invention.

In an exemplary embodiment of the present invention, as a result of performing a survival analysis, it was confirmed that there is a significant association between a significant progression-free survival rate and an increase in overall survival rate in patients with high expression of CXCL13 (see Example 5).

Through the results, the composition including the CXCL13 gene or the protein encoded by the gene of the present invention may be usefully used for predicting the survival rate prognosis of a patient with lung cancer.

As yet another aspect of the present invention, the present invention provides a use of the composition for predicting an anticancer drug treatment response.

As yet another aspect of the present invention, the present invention provides a use of the composition for predicting the survival rate prognosis of a patient with lung cancer.

Hereinafter, preferred examples for helping the understanding of the present invention will be suggested. However, the following examples are provided only to more easily understand the present invention, and the contents of the present invention are not limited by the following examples.

### EXAMPLES

### Example 1

### Experiment Preparation

1-1. Selection of Patients for Providing Clinical and Histological Information

Samples were obtained retrospectively and prospectively from histologically confirmed patients with non-small cell lung cancer treated with PD-1 or the PD-L1 inhibitors pembrolizumab, nivolumab, or atezolizumab. Patients having samples available for whole transcriptome sequencing (WTS) were included in the analysis. The results of 63 patients showing WTS result values using an access kit were used as a search data set. As a verification set, the results of 57 patients showing WTS result values were used using the TruSeq kit. Clinical information was collected from electronic medical records. PD-L1 immunohistochemistry (IHC) results were recorded based on the tumor proportional score (TPS) using the 22C3 pharmDx antibody (Agilent, United States). Pathologists examined samples available for H&E staining. The present invention was conducted with the approval of the Institutional Review Board (IRB number: 2018-03-130).

1-2. Genomic RNA Preparation and Whole Transcriptome Sequencing (WTS)

RNA was purified from formalin fixed paraffin embedded (FFPE) or fresh tumor samples using the ALLPrep DNA/RNA Mini Kit (Qiagen, United States). The concentration and purity of RNA were measured using NanoDrop and Bioanalyzer (Agilent, United States). A library was prepared using TruSeq RNA library Prep Kit v2 and TruSeq RNA Access Library Prep Kit (Illumina). Total RNA isolated by reverse transcription reaction with poly(dT) primers was used using SuperScript TII Reverse Transcriptase (Invitrogen/Life Technologies, USA). A RNA-seq library was prepared by cDNA amplification, end repair, 3′ end adenylation, adapter ligation and amplification. The quality and quantity of the library were measured using Bioanalyzer and Qubit. Sequencing was performed using the Hiseq 2500 platform (IIlumina). Reads for FASTQ files were mapped to an hg human genome using a 2-pass mode of STAR ver-2.4.0. It was confirmed that STAR 1-pass performed reading for the hg19 genome standard and sample-specific alignment using the hg19 genome, and STAR 2-pass attained the newly generated hg19 genome and performed sample-specific alignment using the genome. RNA-SeQC was performed to measure the quality of a bam file. The raw read count mapped to the gene was analyzed for the transcript abundance ratio using RSEM ver-1.2.18, and samples which were erroneously expressed based on the criteria of a read count <1M were removed.

1-3. Calculation of Tumor Purity and Tumor Mutation Burden (TMB) by Differentially Expressed Gene (DEG)

A differentially expressed gene (DEG) analysis was performed among patients exhibiting a partial response (PR) and stable disease (SD) or progressive disease (PD) as assessed by response criteria in solid tumor v1.1 using the Mann-Whitney test. The differentially expressed gene analysis was performed based on a transcript per million (TPM) value of an immune-related gene. A 1.5-fold difference in expression between the groups analyzed by the nominal double-sided Mann-Whitney (P-value<0.05) was used as a cut-off value for significance. The tumor purity was calculated using ESTIMATE. To measure TMB, the library was calculated with SureSelectXT Human All Exon V5 and sequencing was performed using the HiSeq 2500 platform (Illumina). A target range for the normal control was 50× and a target range for the tumor sample was 100×. Sequence analysis data was aligned to the hg19 human genome. Mutations were annotated using Mutect for somatic mutations. TMB was measured by the total number of mutations per Mb except for the same mutation.

1-4. Confirmation of Gene Set of Interest and ICI Response Data from Other Populations

Various gene sets known as the gene expression marker-related gene sets of tumor-infiltrating lymphocytes reported from Cytolytic activity, Immunoscore, cytolytic (CYT) score, gene expression profile (GEP) and Danaher et al. were used. The results of renal cell carcinoma and melanoma were used to adopt the study results on other populations with different types of cancer.

1-5. The Cancer Genome Atlas (TCGA) Data

WTS data for normal lung and lung adenocarcinoma was obtained at Broad GDAC Firehose Level 3 (https://gdac.broadinstitute.org). Clinical data was obtained from cBioPortal (http://www.cbioportal.org). A total of 515 tumor samples and 59 normal samples were available for analysis. The expression profiles of CXCL13 and immune-related genes were compared using the T-test method. A survival analysis was performed based on available clinical information.

1-6. Statistical Analysis

The Kaplan-Meier survival curve was used to understand patterns of progression-free survival (PFS) and overall survival (OS). A P-value was calculated using a log rank test. The correlation was examined using the Pearson correlation method, the differences between two groups were compared using the Mann-Whitney test method, and the differences among three groups were compared using the Kruskal-Wallis test method. All statistical analyses were performed using the R-3.6.0 program, and P-values less than 0.05 were considered to be significant.

### Example 2

### Confirmation of Characteristics of Selected Patients

The basic demographic profile showed similarities between search data and verification data sets. In the search data set, the majority of patients were treated with pembrolizumab (69.4%), nivolumab (36.5%) and atezolizumab (12.7%), and similar patterns were shown in patients in the verification data set for pembrolizumab (49.1%), nivolumab (31.6%), and atezolizumab (7.0%). Treatment was primarily tertiary or more and applied to the search data set (58.7%) and the verification data set (66.7%). The samples used for WTS were usually obtained from lung parenchymal tissue (33.3%) and lymph nodes (LN) (69.4%) for search data sets and lungs (35.1%) and lymph nodes (21.1%) for verification data sets. It was confirmed that patients with high PD-L1 expression defined by TPS PD-L1 IHC as 50% or more accounted for 36.5% in the search data set and 42.1% in the verification data set.

### Example 3

### Confirmation of CXCL13 Upregulation of Search Data Set in ICI Patients

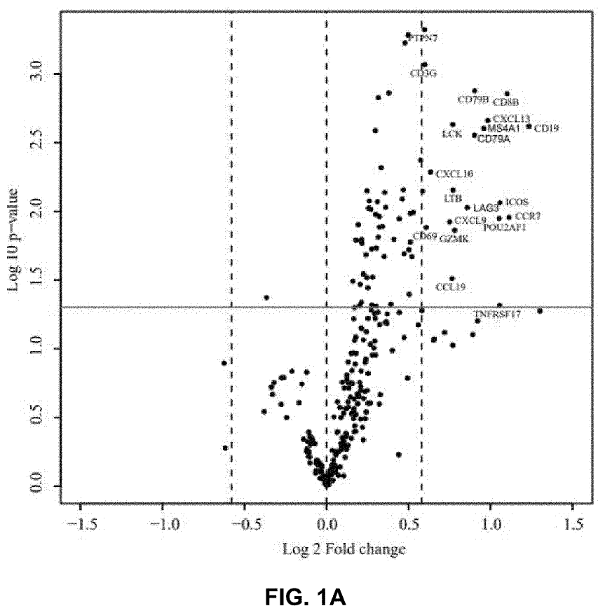

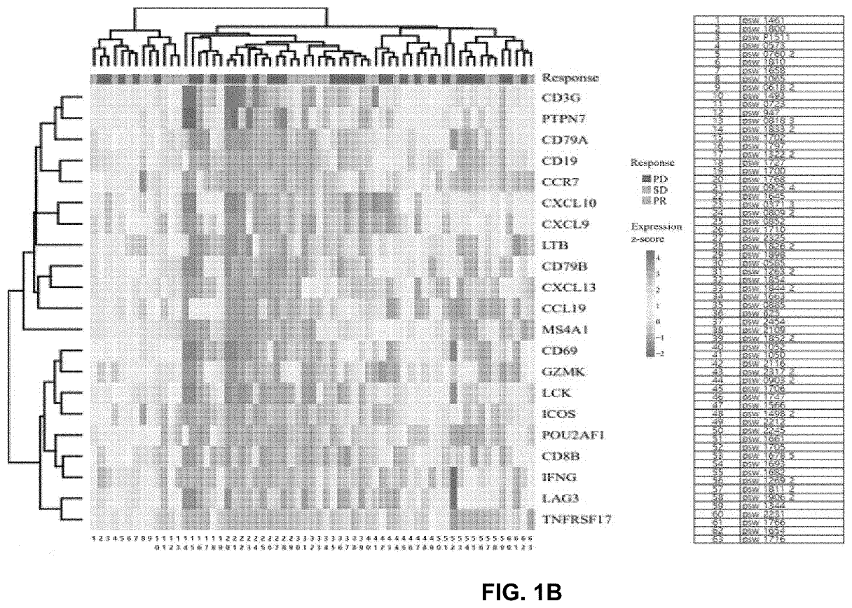

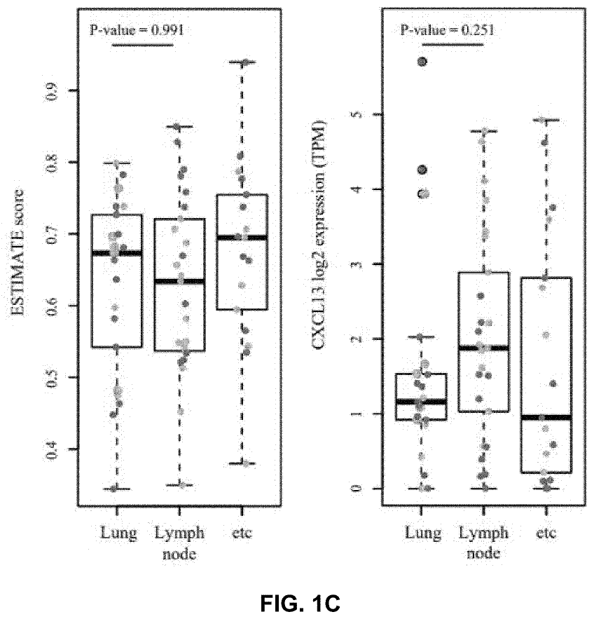

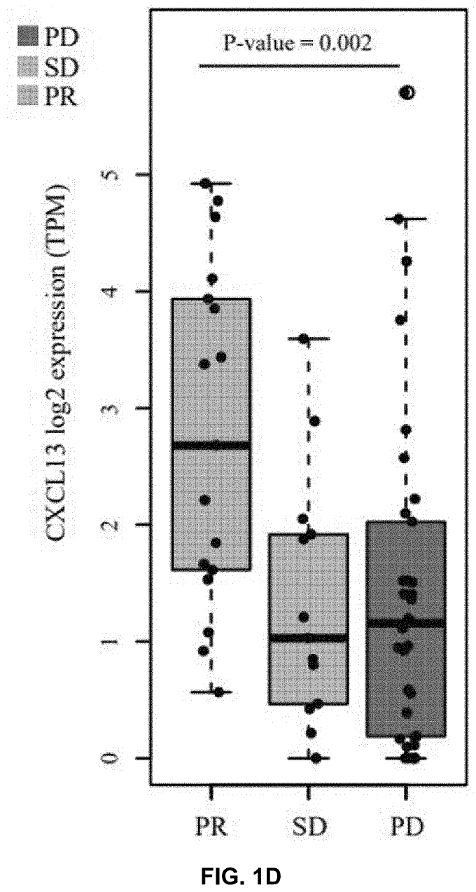

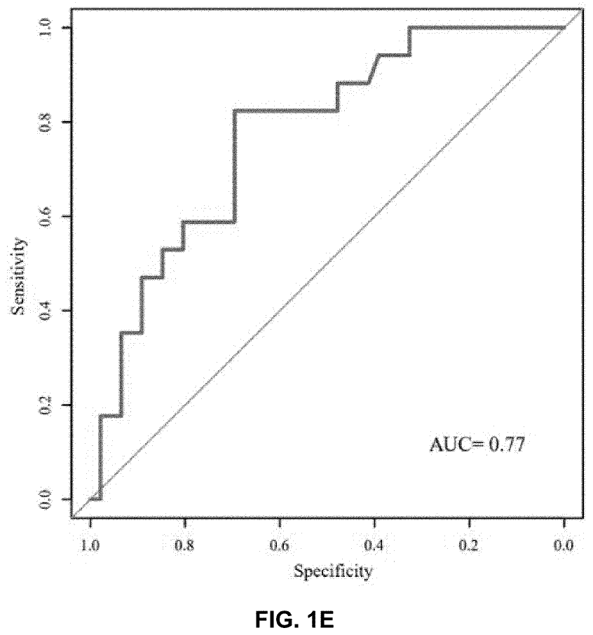

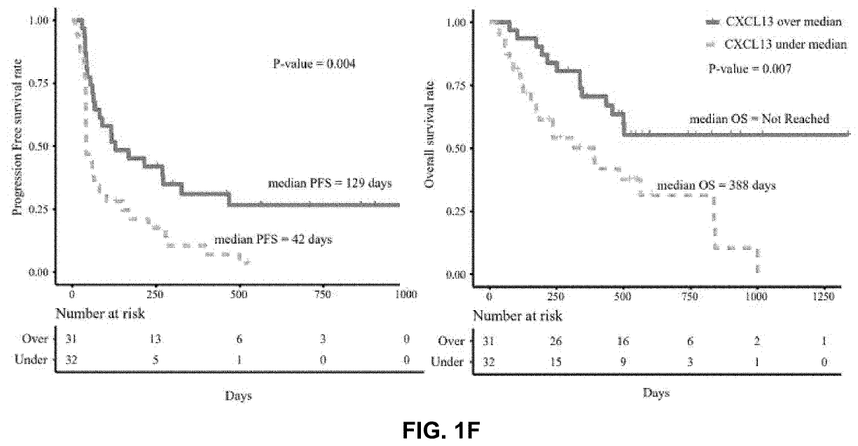

As a result of comparing the differently expressed gene (DEG) profile with the non-response group to confirm the CXCL13 upregulation of the search data set in ICI patients, as illustrated in FIG. 1A, it was confirmed that 21 genes were upregulated in the response group, and as illustrated in FIG. 1B, it was confirmed that CXCL13 showed a 1.97-fold change (P=0.002), and the heatmap showed a higher expression profile tendency in partial response (PR) patients.

Furthermore, as a result of additionally analyzing biopsy sites based on prior studies which confirmed the relationship between CXCL13 and immune cells, as illustrated in FIG. 1C, it was confirmed that there was no difference in the tumor purity calculated by ESTIMATE between the lungs and the lymph nodes (P=0.991).

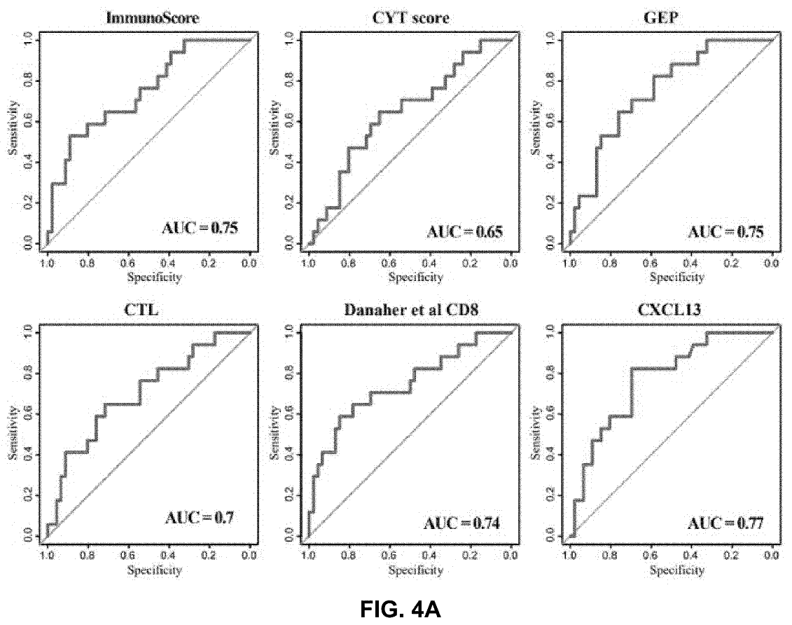

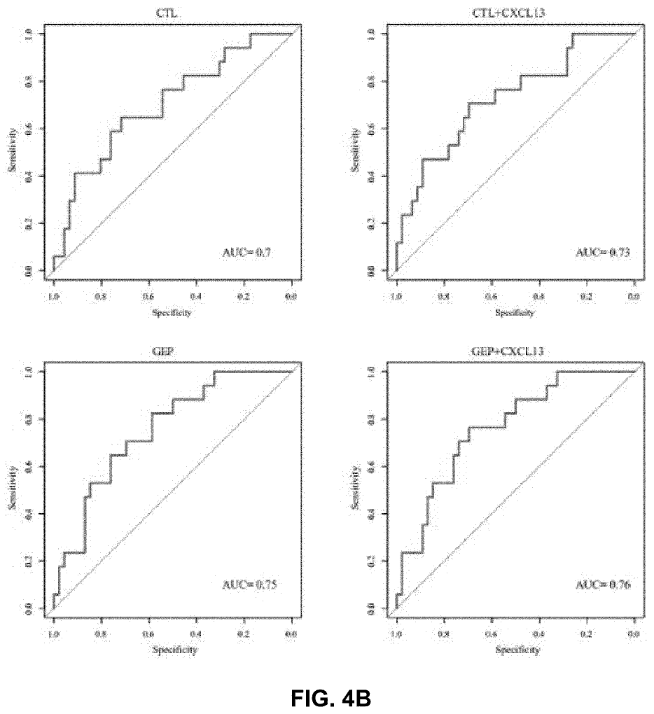

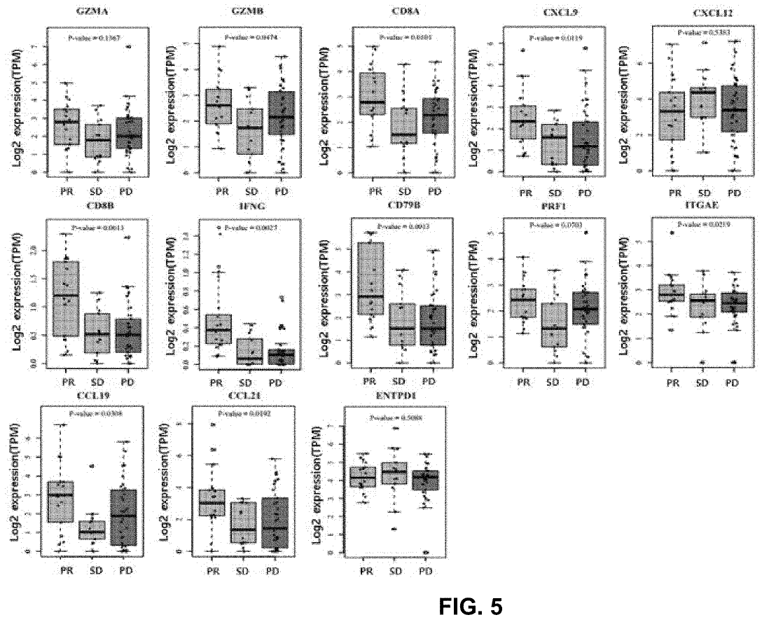

Through the results, it was confirmed that the biopsy sites could be additionally analyzed by excluding the potential bias at the sampling site (P=0.251). Further, as illustrated in FIG. 1D, in partial response (PR) patients, the median TPM value of CXCL13 was 5.41 (95% confidence interval (CI) of 0.48 to 29.38), which is a remarkably higher value than a TPM median value of 1.04 (95% CI of 0.00 to 11.07) in patients with stable disease (SD) and a TPM median value of 1.28 (95% CI of 0.00 to 51.10) in patients with progressive disease (PD). As illustrated in FIG. 1E, it was confirmed that the area under curve (AUC) value was 0.77 when any cut-off value was used as the TPM median value of CXCL13. As a result of comparing the AUC with those of other gene sets from the results, as illustrated in FIG. 4A, results similar to the AUC values obtained for other gene sets such as Immunoscore (AUC=0.75), CYT score (AUC=0.65), GEP (AUC=0.75), CTL (AUC=0.70), and Danaber were confirmed, and as illustrated in FIG. 4B, it was confirmed that the predicted value was not increased when CXCL13 was combined with other gene sets.

In addition, as a result of a survival analysis, as illustrated in FIG. 1F, it was confirmed that PFS (P=0.004) and OS (P=0.007) were considerably longer in the case of a patient with high CXCL13 expression.

### Example 4

### Confirmation of Correlation Between CXCL13 and Other Immune-Related Biomarkers

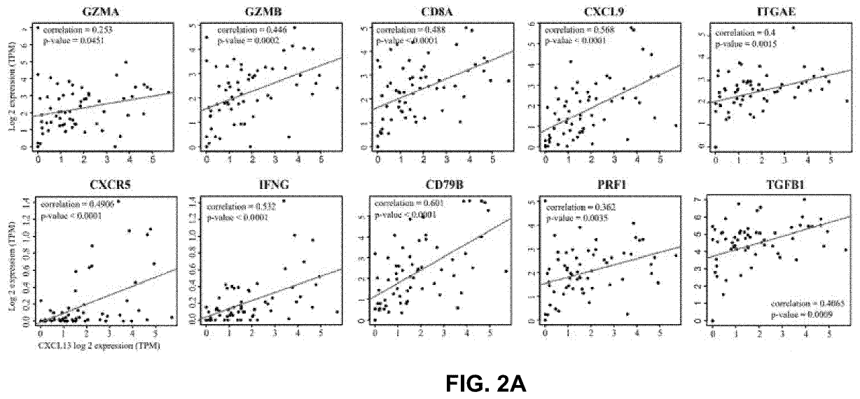

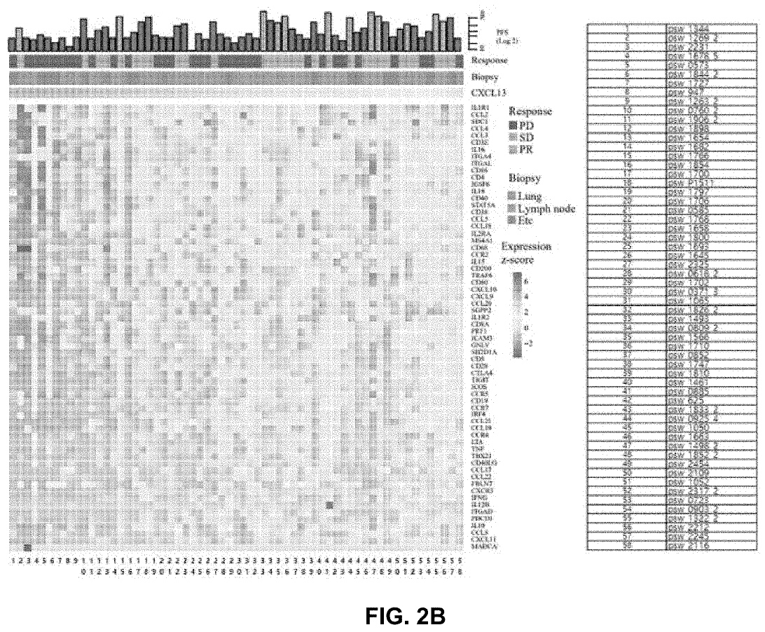

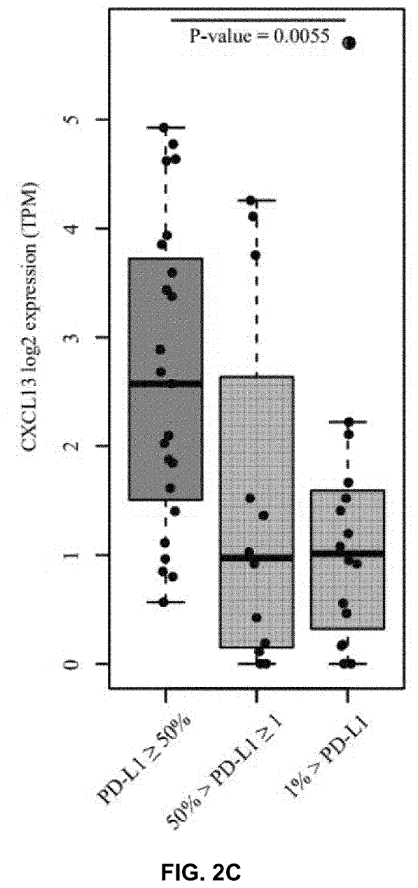

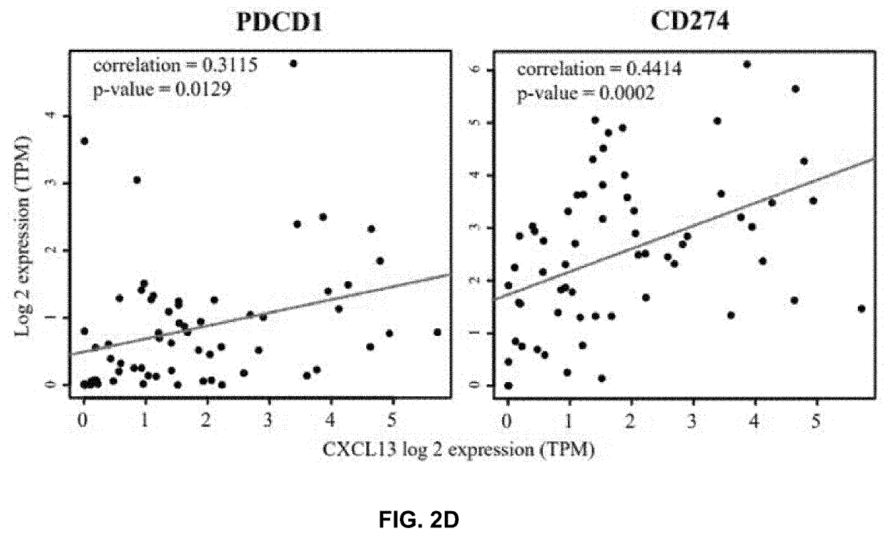

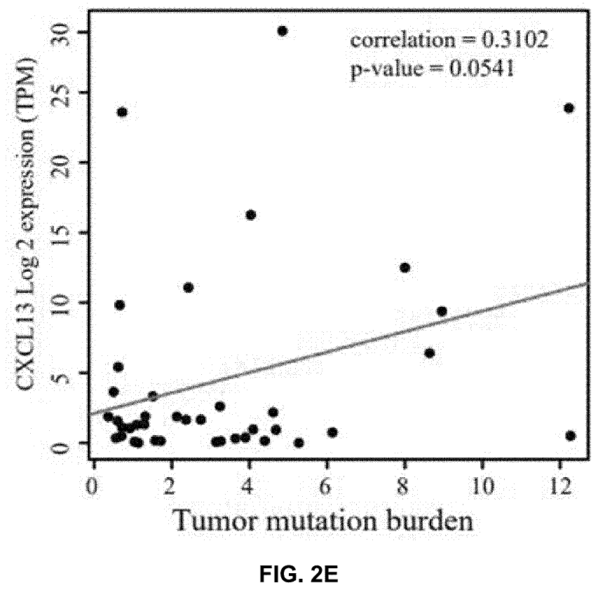

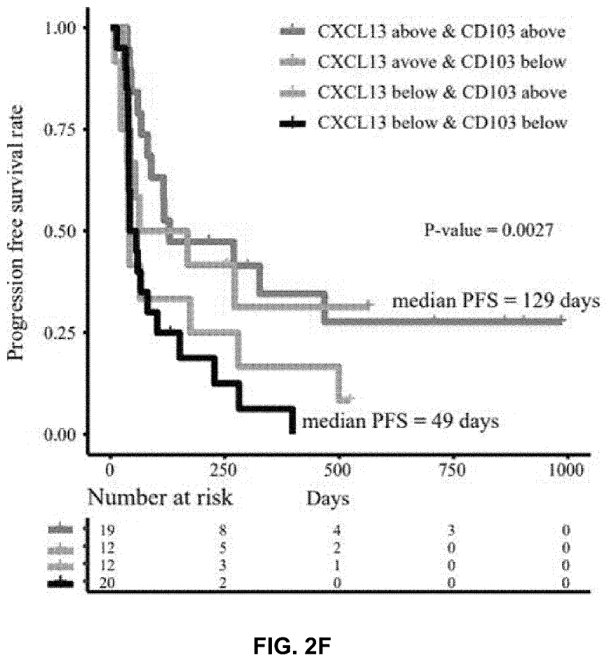

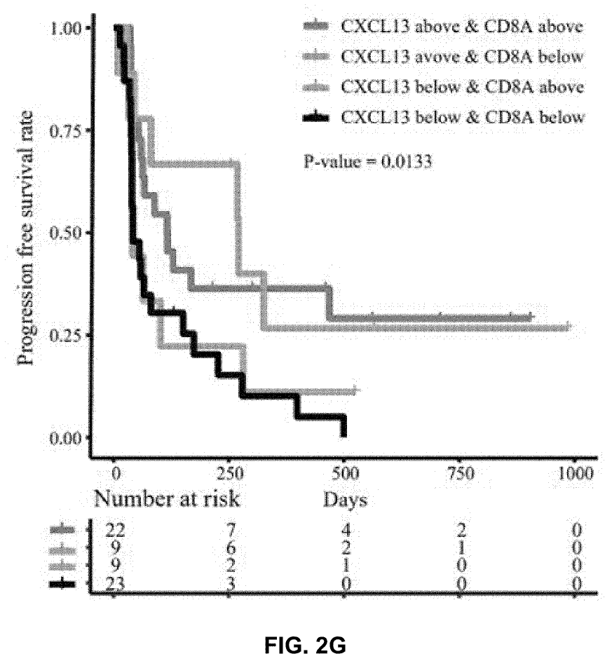

In the search data set, as a result of performing an additional analysis on the correlation between CXCL13 expression and other immune-related genes, as illustrated in FIG. 2A, it was confirmed that representative genes known to be associated with activated cytotoxic T cells showed a correlation with the amount of CXCL13, and as illustrated in FIG. 2B., it was confirmed that CXCL13 showed an expression pattern similar to the gene set of TLS previously reported.

In addition, as a result of performing an additional analysis based on PD-L1 protein expression, as illustrated in FIG. 2C, it was confirmed that the expression of CXCL13 was further improved in samples with PD-L1 50% (P=0.006) compared to PD-L1<50%.

Furthermore, as illustrated in FIG. 2D, the correlation between CXCL13 expression and PD-1/PD-L1 was examined at the transcription level exhibiting a positive tendency with PDCD1 (P=0.013) and CD274 (P<0.001). As a result, as illustrated in FIG. 2E, it was confirmed that the correlation between CXCL13 and TMB was not significant based on the samples (n=41) available for the TMB analysis (P=0.054). Further, as illustrated in FIG. 2F, as a result of analyzing the survival of the subgroup based on the TPM expression median values of CD103 and CXCL13, it was confirmed that CXCL13 played a major role in determining the response to an ICI.

In addition to the results, as a result of analyzing the survival of the subgroup based on the TPM expression median values of CXCL13 and CD8A, as illustrated in FIG. 2G, it was confirmed that when CXCL13 and CD8A were upregulated, CXCL13 and CD8A exhibited a significant PFS prolongation compared to the low subgroup (P=0.013).

### Example 5

### Confirmation of Results in Verification Data Sets and Other Populations

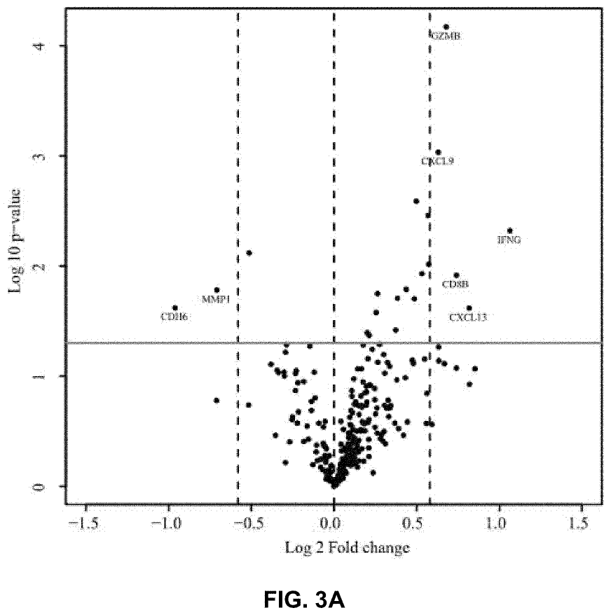

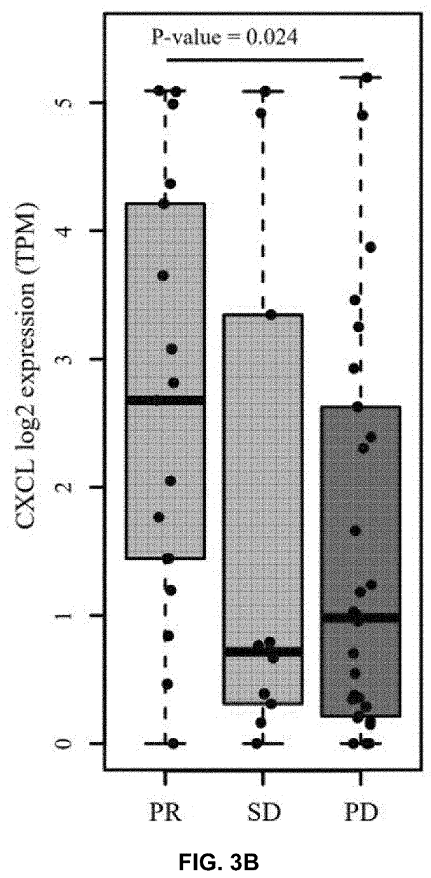

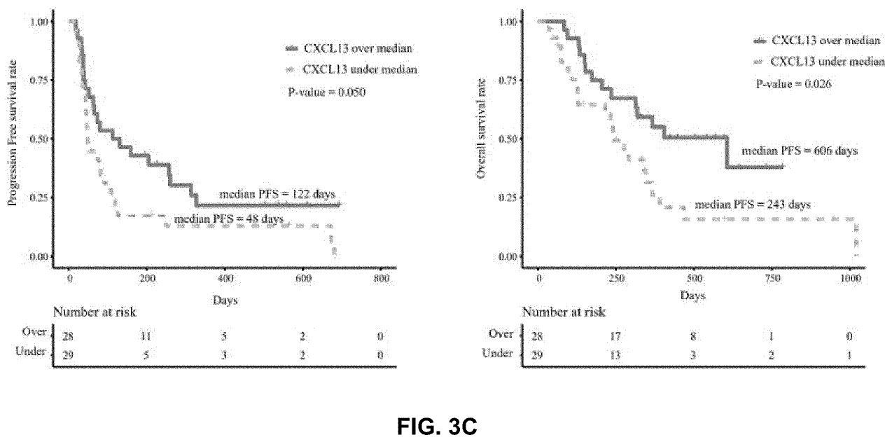

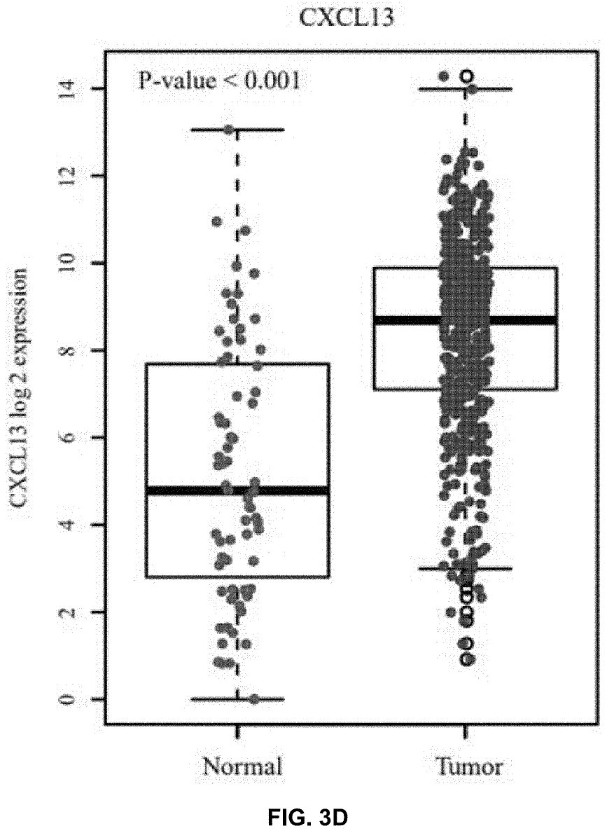

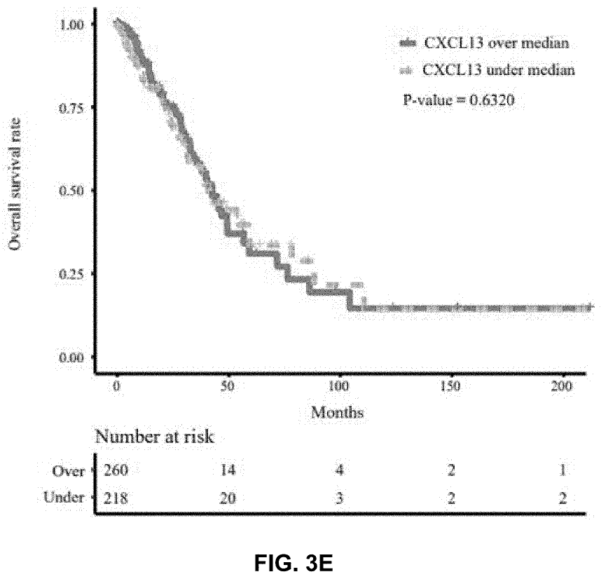

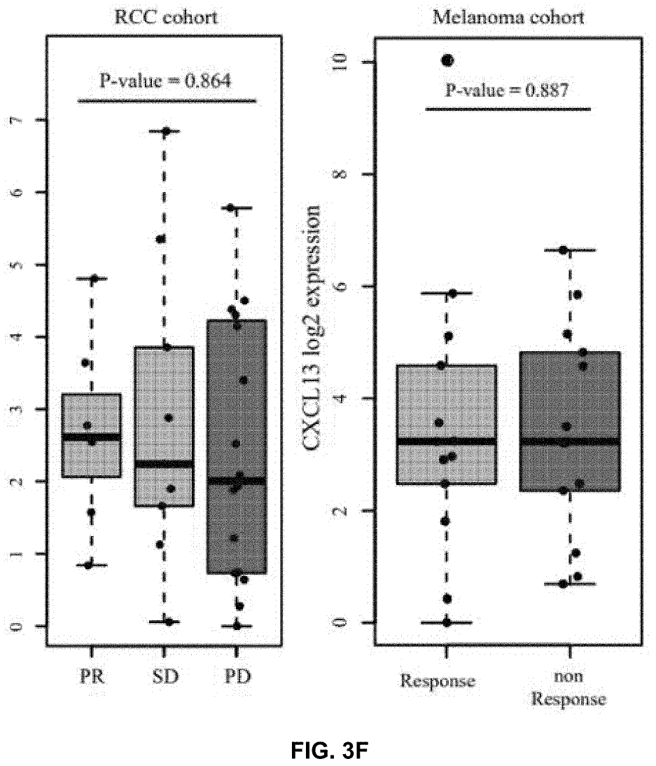

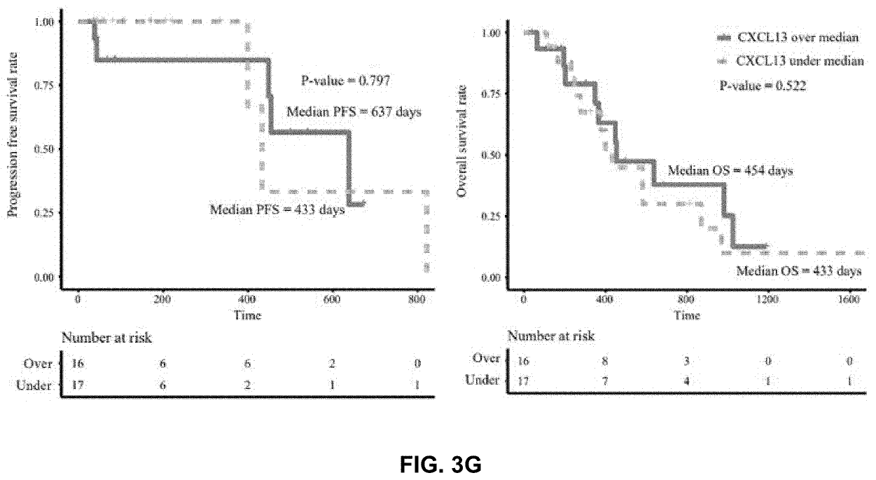

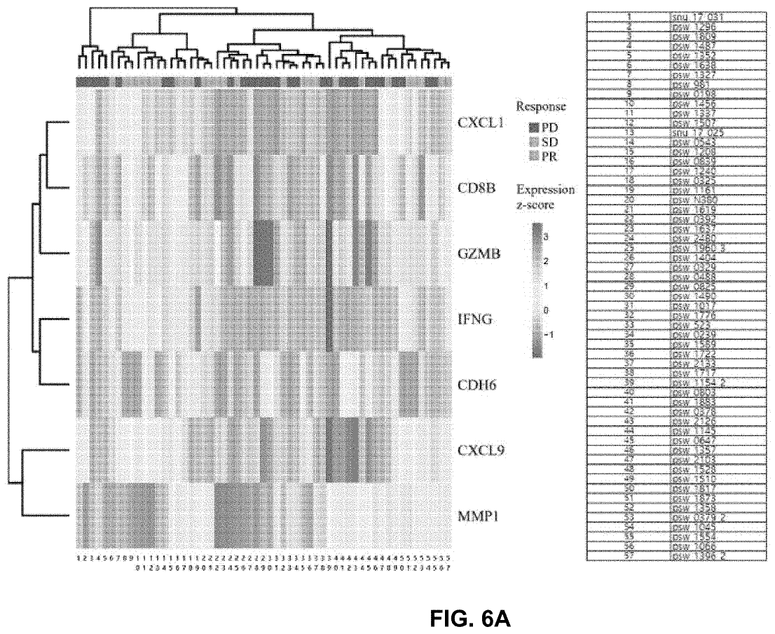

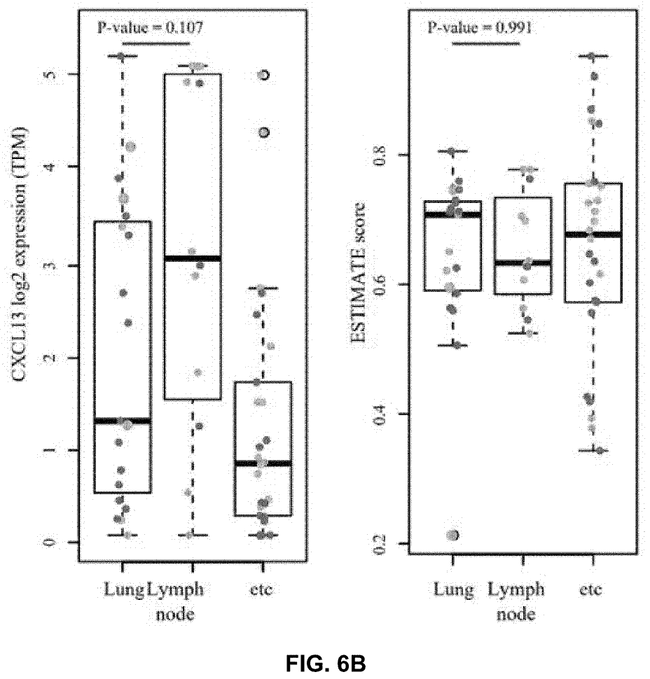

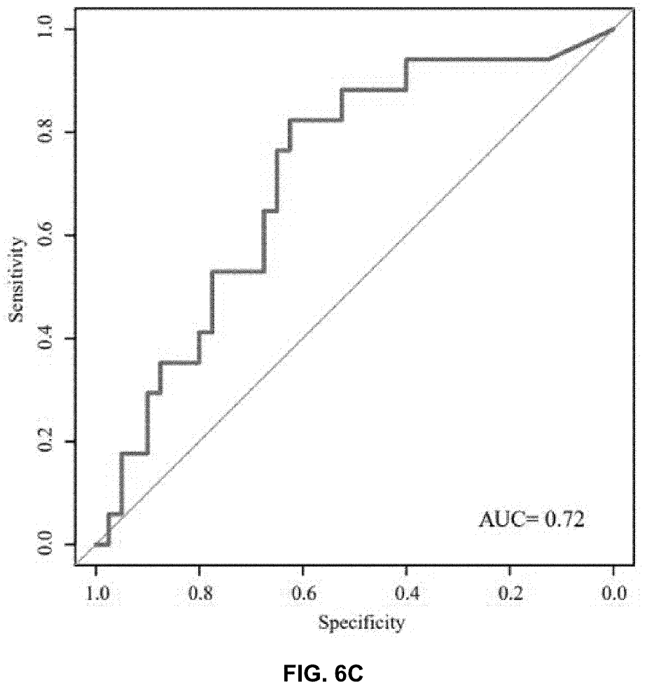

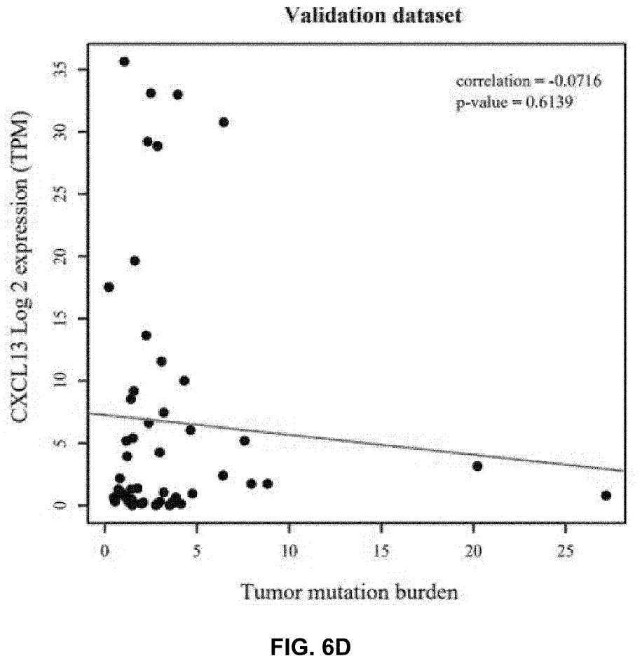

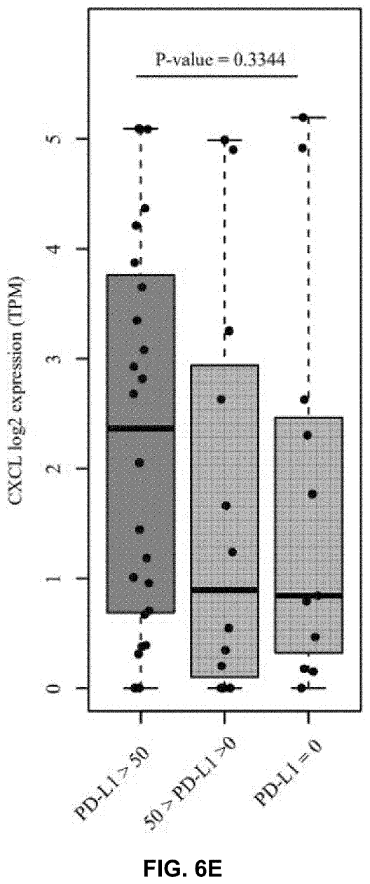

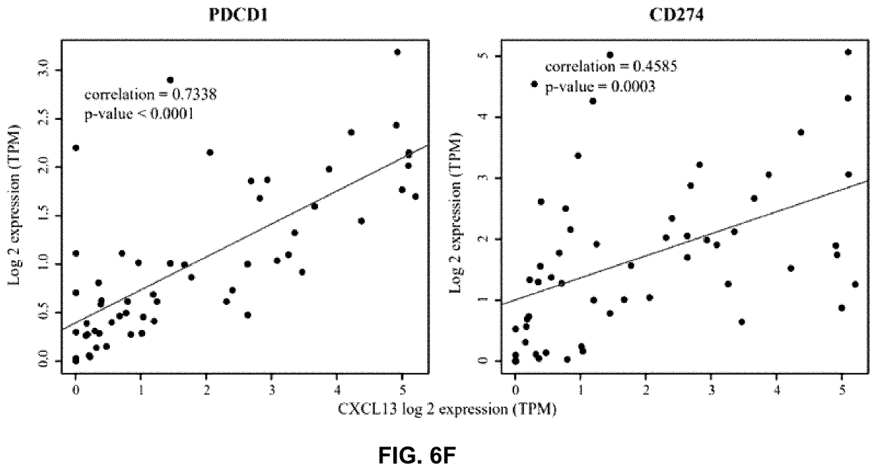

A similar examination was performed based on the response to an ICI in the verification data set (n=57), and as a result of confirming a significant difference in 7 genes CXCL13, CD8B, IFNG, CDH6, CXCL9, and MMP1 in DEG, as illustrated in FIGS. 3A, 3B, and 6A, CXCL13 showed a 1.76-fold increase (P=0.024) in responders.

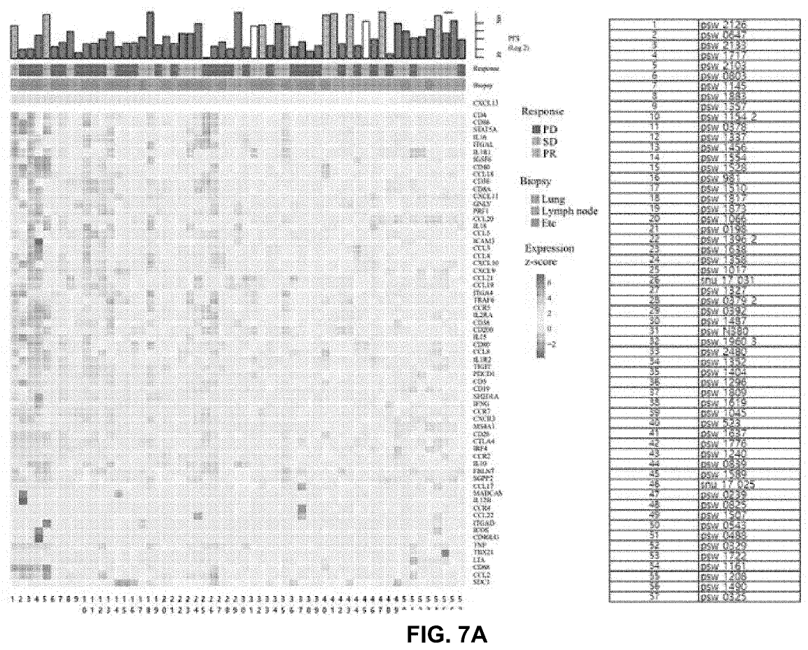

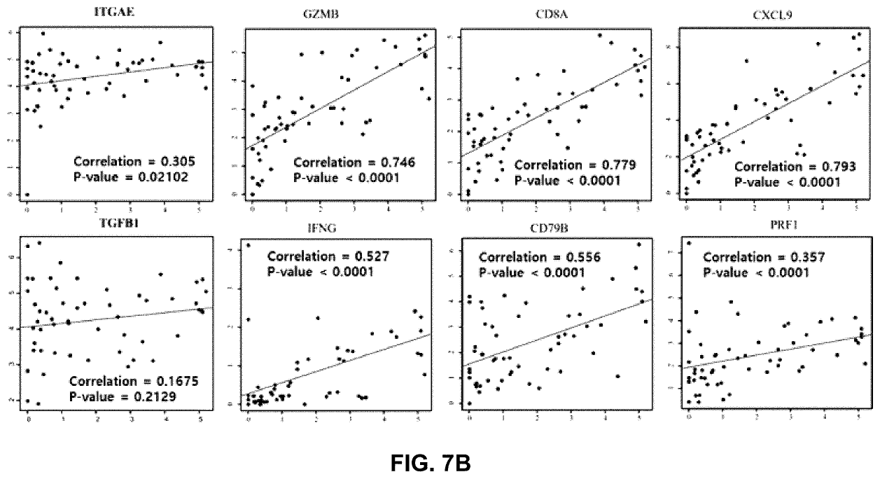

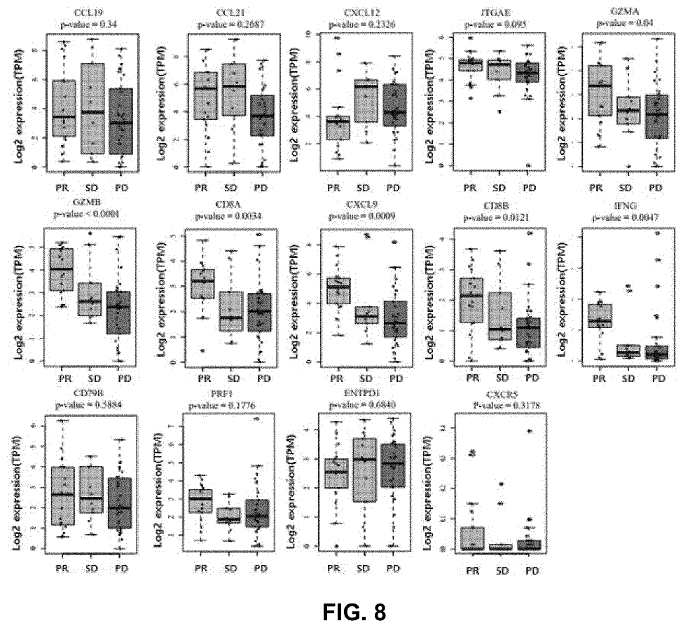

In addition, as a result of performing a survival analysis, as illustrated in FIGS. 3C and 6C, in patients with high CXCL13 expression and similar predicted values (AUC =0.72), remarkably prolonged PFS (P=0.050) and OS (P=0.026) values were confirmed. Further, as illustrated in FIGS. 7A, 7B, and 8, similar patterns related to TMB (P=0.614) for immune-related genes and TLS-related gene sets were confirmed.

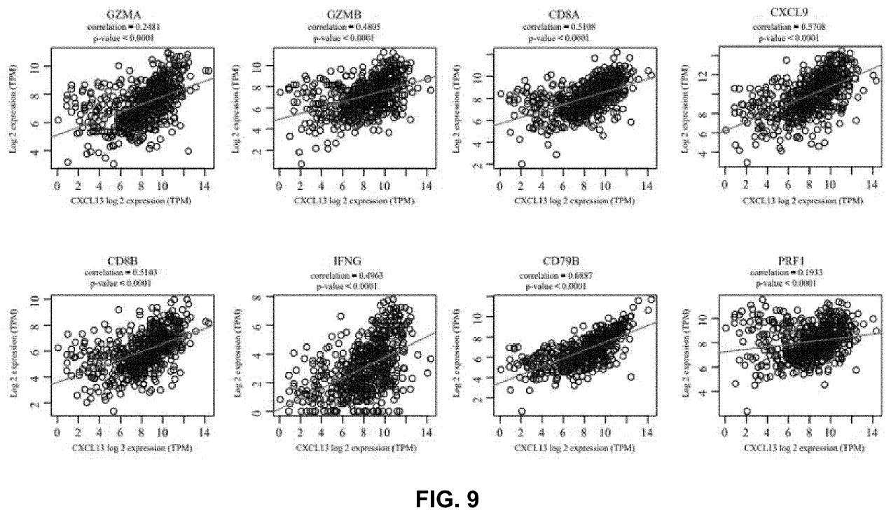

Furthermore, an additional analysis was performed using ADC samples of TCGA data. As a result of comparing the normal samples and the tumor samples, as illustrated in FIG. 3D, CXCL13 was significantly upregulated in the tumor samples (P<0.001), and as illustrated in FIG. 9, it was confirmed that the expression of CXCL13 in the tumor samples showed a positive correlation with a representative cell lysis activity-related gene. Since most treatments in the TCGA population are cytotoxic agents, there was no difference in the CXCL13 expression profile in OS values, as illustrated in FIG. 3E (P=0.632).

Furthermore, as a result of examining publicly available renal cell carcinoma (RCC) and melanoma populations treated with ICIs to confirm similarity in other carcinomas, as illustrated in FIGS. 3F and 3G, it was confirmed that CXCL13 did not exhibit characteristics of prediction for response or for increased survival in the two populations.

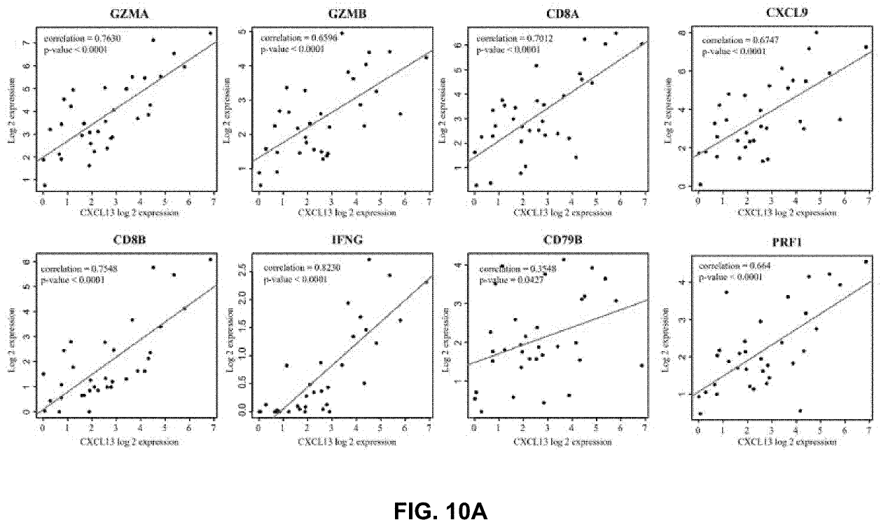

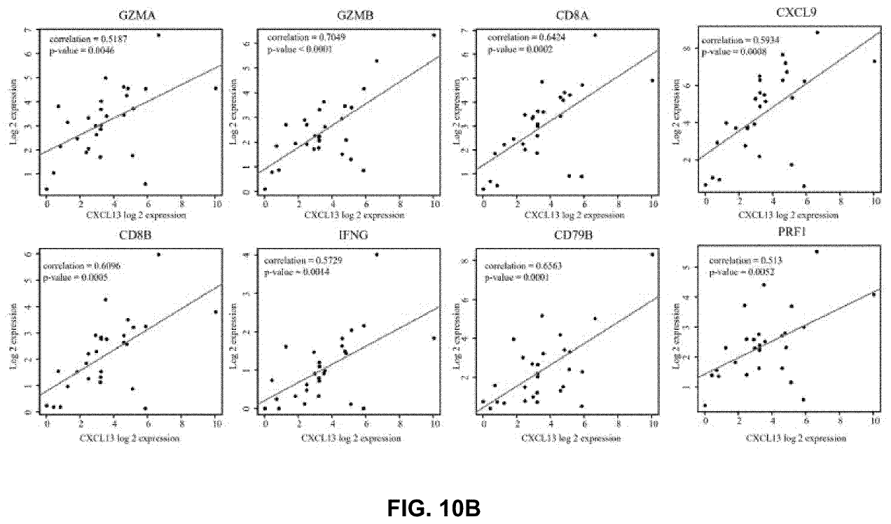

In addition, as illustrated in FIG. 10A, the correlation between CXCL13 expression in the renal cell carcinoma population and representative genes associated with independent cytotoxic activity was confirmed, and as illustrated in FIG. 10B, the correlation between CXCL13 expression in the melanoma cell population and representative genes associated with independent cytotoxic activity was confirmed.

### Example 6

### Confirmation of Presence of Tertiary Lymphatic Structure and Correlation with CXCL13 in NSCLC Adenocarcinoma

As a result of confirming the correlation between the CXCL13 expression profile and TLS confirmed in a histological slide by manually examining H & E slides in order to assess a histological correlation with transcript data, it was confirmed that the expression of CXCL13 was high in the tertiary lymphatic structure adjacent to the tumor.

The above-described description of the present invention is provided for illustrative purposes, and those skilled in the art to which the present invention pertains will understand that the present invention can be easily modified into other specific forms without changing the technical spirit or essential features of the present invention. Therefore, it should be understood that the above-described embodiments are only exemplary in all aspects and are not restrictive.

### Industrial Applicability

It was confirmed that CXCL13 was significantly upregulated in a patient administered an immune checkpoint inhibitor using the composition according to the present invention, so that it is expected to be widely utilized as a new immunoadjuvant to enhance the anticancer immunotherapeutic effect in a patient with lung cancer by regulating the level of mRNA of a CXCL13 gene or a protein encoded by the gene.

1-17. (canceled)

18. A method for predicting immunotherapeutic responsiveness in a patient with lung cancer, the method comprising measuring the expression level of mRNA of a CXCL13 gene or a protein encoded by the gene for a subject-derived biological sample.

19. The method of claim 18, wherein the immunotherapy is treatment with a PD-1 inhibitor or a PDL-1 inhibitor.

20. The method of claim 18, wherein the lung cancer is lung adenocarcinoma.

21. The method of claim 18, wherein the expression level of mRNA is measured by one or more methods selected from the group consisting of NanoString nCounter analysis, a polymerase chain reaction (PCR), a reverse transcription-polymerase chain reaction (RT-PCR), a real-time polymerase chain reaction (Real-time PCR), RNase protection assay (RPA), a microarray, and northern blotting.

22. The method of claim 18, wherein the expression level of the protein is measured by one or more methods selected from the group consisting of western blotting, radioimmunoassay (RIA), radioimmunodiffusion, enzyme-linked immunosorbent assay (ELISA), immunoprecipitation, flow cytometry, immunofluorescence, Ouchterlony double immunodiffusion, complement fixation assay, and a protein chip.

23. The method of claim 18, wherein the biological sample is a tissue derived from a patient with lung cancer.

24. A kit for predicting immunotherapeutic responsiveness in a patient with lung cancer, comprising a preparation which measures the level of mRNA of a CXCL13 gene or a protein encoded by the gene.

25. The kit of claim 24, wherein the immunotherapy is treatment with a PD-1 inhibitor or a PDL-1 inhibitor.

26. The kit of claim 24, wherein the lung cancer is lung adenocarcinoma.

27. The kit of claim 24, wherein the preparation which measures the level of mRNA of the gene is a set of sense and antisense primers or a probe which binds complementarily to the mRNA of the gene.

28. The kit of claim 24, wherein the preparation which measures the level of the protein is an antibody which binds specifically to the protein encoded by the gene.

29. A method for screening an immunotherapeutic adjuvant, the method comprising:
(a) treating cells in vivo with a candidate material;
(b) selecting a candidate material in which the expression of m RNA of a CXCL13 gene or a protein thereof is increased compared to a candidate material non-treatment group by measuring the expression level of mRNA of the CXCL13 gene or the protein thereof; and
(c) selecting the selected candidate material as an immunotherapeutic adjuvant.

30. The method of claim 29, wherein the candidate material is selected from the group consisting of nucleic acids, compounds, microbial culture solutions or extracts, natural product extracts, peptides, substrate analogues, aptamers, and antibodies.
